(19) United States
(12) Patent Application Publication
(10) Pub. No.: US 2004/0247681 A1
(43) Pub. Date: Dec. 9, 2004

(54) Drug release system for controlled therapy

(76) Inventors: Edward J. Ellis, Lynnfield, MA (US); Jeanne Y. Ellis, Lynnfield, MA (US)

Correspondence Address: CANTOR COLBURN, LLP, 55 GRIFFIN ROAD SOUTH, BLOOMFIELD, CT, 06002

(21) Appl. No.: 10/486,130

(22) PCT Filed: Aug. 7, 2002
(86) PCT No.: PCT/US02/25040

Related U.S. Application Data
(60) Provisional application No. 60/310,507, filed on Aug. 7, 2001.

(51) Int. Cl. A61K 9/14

(52) U.S. Cl. 424/486

(57) ABSTRACT

Polymeric compositions containing a high percentage of bound polypropylene glycol provide matrices for the controlled release of drugs and medicinal agents. The compositions are prepared by the polymerization of ethylenically unsaturated polypropylene glycol containing monomers. Copolymers of ethylenically unsaturated polypropylene glycol containing monomers with co-monomers are also disclosed. The drug loaded polymeric compositions of this invention find particular utility as ocular insets for the controlled release of drug(s) into the eye.

### STATEMENT REGARDING FEDERAL SPONSORSHIP

This invention was made with government support under grant no. 1 R43 EY13479-01 awarded by the National Institute of Health. The government has certain rights in the invention.

### TECHNICAL FIELD

This invention relates to a composition, a method and a device for the controlled administration of therapeutically active agents. More particularly, this invention relates to drug dispensing devices, which are each classified as a “Matrix System.” In preferred embodiments, the invention relates to a matrix system for the controlled and continuous administration of drug to a mammalian patient, especially to the eye of such patient over a prolonged period of time. Another aspect of the invention relates to a method of preparing these devices.

### BACKGROUND

Many and varied compositions, products, appliances, depositors, applicators, dispensers and injectors are well known in the art in which the timing or spacing administration or absorption of drug is regulated by the structure or physical arrangement of elements so that a single administration provides a gradual but sustained feeding of the drug to a patient by slow or differential release. The advantages of such devices are that they enable the physician the more carefully regulate the level of drug administration to the patient. A further advantage of sustained release devices is the fact that the number of times that the drug need be administered is reduced.

Where oral administration is desired, one means for obtaining the above objective is to employ capsules or tablets which release the drug at a uniform rate during the capsule's passage through the gastrointestinal tract. In the past this object has been achieved by admixing one or more inert ingredients with the drug in such a manner that these inactive materials interfere with the disintegration of the tablet or the dissolution of the drug. One form of such a tablet is one wherein tablets can be composed of several alternate layers of medicament and inert material. In this manner, as each alternate protective layer disintegrates the patient receives a further dose of medicament. However, tablets of this type suffer from the disadvantage of not providing a uniform and constant drug release. Furthermore, such tablets are difficult to prepare with precision so that in many instances the desired dosage level cannot be assured. Moreover, it has not been possible to provide for prolonged release of a drug by these tablets because of their rapid rate of degradation or dissolution. Still further, degradable carriers of this type have not generally found wide acceptance because of the undesirable side effects which they often produce, for example, foreign body reaction and scar formation.

Recognizing these disadvantages, more recently, there have been developed certain synthetic polymeric carriers, most notably polysiloxane rubbers, which are designed to deliver a drug to the patient without concomitant degradation of the delivery device. Instead, the polymeric drug delivery systems are based upon the phenomenon of diffusion in which drug migrates through a polymer wall at a relatively low rate. In such a system, the drug is disposed throughout the polymeric carrier manufactured from the polymeric material.

At the present time, drugs of various kinds are frequently employed in ophthalmic practice for the treatment of eye diseases as well as infections and inflammation. Since these drugs are rapidly excreted from the body or diffuse from any site of local application, repeated or numerous administration of the drug during the crucial period is generally necessary. Therapeutic substances may be introduced into the eye by various methods. The methods generally used are instillation in the conjunctival sac in the form of drops or ointments. In this method, drugs enter the eye largely through the cornea but to be effective, in many cases, the application of the drug must be substantially continuous. At the present time, it is not possible to obtain continuous delivery of a given drug through the use of drops or ointments even though they are applied at intervals during a given period. Periodic application of such dosage forms generally results in the eye receiving a large but uncertain amount of the drug at the moment it is applied, but the drug is washed away rapidly by tears, thus leaving the eye without medication until the next application. For example, persons suffering from glaucoma, a symptomatic condition characterized by an increase in intraocular pressure, must use eye drops in large quantities and at frequent intervals in order to maintain the base pressure below a reasonable level. Timolol is generally used in the treatment of glaucoma, but frequent administration is required due to the fact that the hypotensive action of the drug is not of long duration.

Thus, there still remains a need to find better methods of delivering drugs to the eye so as to obtain the maximum effect from the drug without the need for frequent administration.

One method which has been proposed for the treatment of acute glaucoma, for example, is to deliver the drug to the eye enclosed in a polyvinyl membrane. The membrane containing the drug is applied to the eyelid. However, it was found that the inclusion of the drug in a membrane did not increase the effectiveness of the drug in the general treatment of acute attacks of glaucoma.

There have been many attempts to construct devices for delivering a drug over a prolonged period of time, generally hours or days to perhaps months. These devices can be divided into two classes: erodible and non-erodible.

Erodible devices are usually polymers that degrade by hydrolysis. As the outer surface layers of the device erode, the included drug is released from the matrix. An example of this type of device is disclosed in U.S. Pat. No. 5,707,643 to Ogura. In some cases a simple dissolution of a water soluble polymer system containing a drug is classified as erodible. An example of this type of device is disclosed in U.S. Pat. No. 5,556,633 to Haddad.

Non erodible devices can take several forms. The reservoir delivery systems are composed of a solid envelope or shell enclosing a liquid, solid or pasty core substance. This solid envelope or shell is a release rate-controlling membrane. A core, such as a drug, is released from the reservoir by diffusion across the wall membrane under concentration gradient of the core between the inside and outside of the device. Examples of this type of device are disclosed in U.S. Pat. No. 3,961,628 to Arnold and U.S. Pat. No. 4,402,695 to Wong.

In contrast to the reservoir systems, matrix (monolithic) systems consist of a “full” solid mass containing a dispersed or dissolved liquid, or solid, drug substance. The drug is released from the matrix system by diffusion of the drug molecules through the matrix under a concentration gradient. An example of this type of device is disclosed in U.S. Pat. No. 4,281,654 to Shell.

Swelling-controlled systems or hydrogels are hydrophilic polymeric networks which can absorb a significant amount of water while maintaining a distinct three dimensional structure when placed in an aqueous solution. Drugs are dispersed or dissolved in the hydrogel network. Once a swelling-controlled system or hydrogel is administered into the body of a patient, water diffuses into the hydrogel, swelling occurs, and the drug molecules diffuse out. An example of this type of device is disclosed in U.S. Pat. No. 4,910,015 to Sung.

Osmotic systems, or osmotic pumps, deliver drugs by pumping drug solution out of the device, driven by osmotic pressure. The osmotic pressure may be generated by the drug molecules or by an added salt or sugar. An osmotic pump is constituted by a rigid polymer case that is semi permeable and a drug powder or a drug solution plus an osmotic agent. Diffusion of water through the semi permeable polymeric membrane or case under osmotic pressure is followed by a convective flow of drug solution under hydrostatic pressure. Examples of this type of device are disclosed in U.S. Pat. Nos. 5,607,696 and 5,609,885 to Rivera.

While much progress has been made in definig prolonged drug release systems and devices, there still remains a need to find better methods of delivering drugs so as to obtain the maximum effect from the drug without the need for frequent administration.

### SUMMARY

The preparation of polymeric products for use in animals and humans is provided herein. More particularly, it is concerned with polymeric matrices or carriers containing therapeutically active substances. Specifically, it is concerned with devices and components comprising a polypropylene glycol based polymer matrix (i.e., a carrier) and therapeutically active agents, which can be used in the treatment of medical disease or disorders. The compositions of this invention are particularly useful in the treatment of diseases or disorders related to the eye.

Surprisingly, polymeric compositions containing a high proportion of polypropylene glycol segments have been found to accept high drug loadings and release those drugs over a prolonged period of time. This provides a number of advantages not found in current drug delivery systems.

The polymeric materials that are used in the present drug delivery carriers are classified as “matrix systems”. For the purpose of the present application, a matrix system is defined as a system that is a controlled release device consisting of a “full” solid mass containing liquid, solid, or pasty active agents. In the matrix system, one or more active agents and the matrix material(s) are mixed together uniformly. The active agent may be dispersed in the matrix, may be dissolved in the matrix, or may be both. The active agents are released from the matrix by molecular diffusion through the matrix (and pores) under a concentration gradient.

Accordingly, one object is to provide a device for the administration of a locally or systemically acting agent to produce a physiologic or pharmacologic effect which also provides technological advancement over prior art devices.

Another object is to provide a dosage regimen for administering an active agent to the eye for a particular time period, the use of which requires intervention only for initiation and termination of the regimen.

Further, another object is to provide an ocular insert which is comfortable to wear for long periods and does not cause discomfort during sleeping and normal daily wear while simultaneously administering drug to the eye.

Yet another object is to provide a drug releasing component which is an integral part of an intraocular lens system.

A process for making such new ocular drug dispensing devices is also provided. Also, the invention provides an ocular device for the controlled release of drug having enhanced mechanical and physical properties.

In one aspect, an ocular delivery device is disclosed for the continuous administration of an active agent over a prolonged period of time comprising ali insert shaped for insertion into the eye. The device is shaped and adapted for insertion and comfortable placement in the cul-de-sac of the conjunctiva between the sclera of the eyeball and the eyelids. Drugs and ocular lubricants are representative active agents for use in this application.

In another aspect, a drug delivery matrix for the continuous administration of an active substance over a prescribed period of time within the eye itself is provided. The delivery matrix, as an exemplary embodiment, is securely attached to an intraocular lens system. Once the intraocular lens is implanted active agent is released into the eye over a prolonged period of time. Drugs such as antibiotic agents and anti-inflammatory agents are representative active substances for use in this application.

Other features and advantages of the invention will be apparent to those skilled in the art from the following detailed description of the invention and the accompanying claims.

### DETAILED DESCRIPTION OF PREFERRED EMBODIMENTS

In accordance with the practice of presently disclosed devices, it has now been unexpectedly found that certain polymeric materials can be used for forming devices for the controlled release of an active agent, such as a pharmaceutical composition (e.g., a drug), for example by diffusion. As used throughout the present application, the term “drug” refers to any number of types of active agents in a number of different forms, such as a pharmaceutical drug.

The use of and advantages realized by the disclosed polymeric materials are unexpected because they can be formulated to accept high levels of drug loading and exhibit release over a prolonged period of time. Furthermore, polymeric materials can be formulated to accept a wide variety of drugs, both hydrophilic and hydrophobic types. The present polymeric materials are compatible with human tissue. That is, these materials do not break down in situ, there is no absorption of the materials, and there is no deleterious action on the sensitive tissues in the area of placement and retention of the system over a prolonged period of time.

The polymers suitable for the purpose of any of the exemplary devices disclosed herein include polymers, copolymers and the like, that are prepared by free radical polymerization and formed into desired shapes by casting or molding.

According to one exemplary embodiment, polymeric materials are disclosed that are suitable as matrices for the controlled delivery of drugs. The polymeric material that form the polymeric matrix contains at least 50% by weight polypropylene glycol segments having the formula:

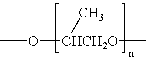

where n=2 to about 100.

The polypropylene glycol segment contains at least one ethylenically unsaturated moiety that can enter into a polymerization reaction and generally has the following structure:

P—Y—

where: P is an ethylenically unsaturated polymerizable group chosen from among

CH₂═CH— or

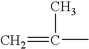

and Y is a spacer group chosen from, but not limited to:

—CO—

—CONH—

—NHCO—

—OCONH—

—CONHCO—

—CONHCONH—

—OCONHCO—

—NHCONHCO—

—NHCONH—

—CH₂—

—CH₂CH₂—

—CH₂CH₂CH₂—

—CH₂CH₂CH₂CH₂—

—C₆H₄—

—C₆H₄CH₂—

—COOCH₂CH(OH)CH₂—

—COOCH₂CH₂—

—COOCH₂CH₂OCH₂CH2— and

—COOCH₂CH₂NHCO—

Examples of ethylenically unsaturated polypropylene glycol compositions include, but are not limited to:

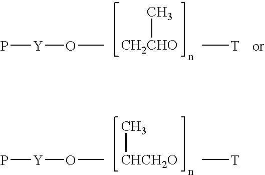

where: P is an ethylenically unsaturated polymerizable group;

Y is a spacer group;

T is a terminal group which is preferably hydrogen or an alkyl group; and

n is an integer from 2 to about 100; or

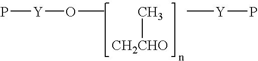

where: P is an ethylenically unsaturated polymerizable group;

Y is a spacer group;

n is an integer from 4 to about 100; or

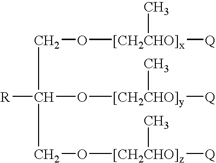

where: Q is independently hydrogen, an alkyl group or P—Y—;

P is an ethylenically unsaturated polymerizable group;

Y is a spacer group;

R is hydrogen or alkyl;

and at least one Q group is P—Y— and x,y and z are independently integers

from 2 to about 100; or

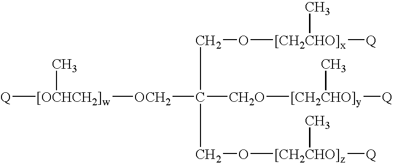

where: Q is independently hydrogen, an alkyl group or P—Y—;

P is an ethylenically unsaturated polymerizable group;

Y is a spacer group;

R is hydrogen or alkyl;

w,x,y and z are independently integers from 2 to about 100;

and at least one Q group is P—Y—; or

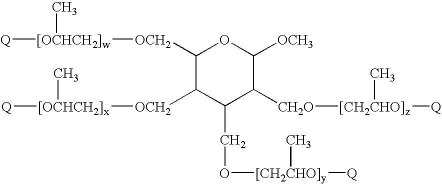

where: Q is independently hydrogen, an alkyl group or P—Y;

P is an ethylenically unsaturated polymerizable group;

Y is a spacer group;

w, x, y and z are independently integers from 2 to about 100;

and at least one Q group is P—Y—; or

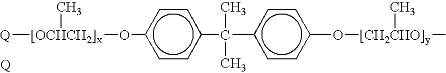

where: Q is independently hydrogen, an alkyl group or P—Y—;

P is an ethylenically unsaturated polymerizable group;

Y is a spacer group;

x and y are independently integers from 2 to about 100;

and at least one Q group is P—Y—.

Exemplary polypropylene glycol containing monomers that are suitable for use in the present devices include:

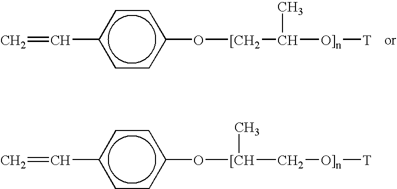

where: T is a terminal group which is preferably hydrogen or an alkyl group;

n is an integer from 2 to about 100; or

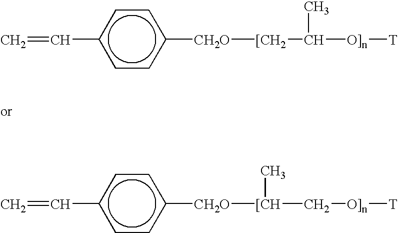

where: T is a terminal group which is preferably hydrogen or an alkyl group;

n is an integer from 2 to about 100; or

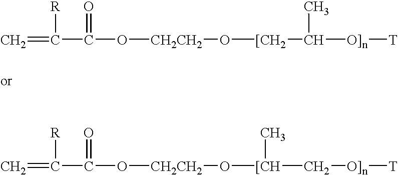

where: R is hydrogen or methyl;

T is a terminal group which is preferably hydrogen or an alkyl group;

n is an integer from 2 to about 100; or

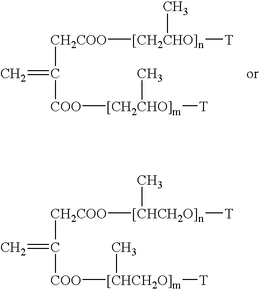

where: T is a terminal group which is preferably hydrogen or an allyl group;

n and m are independently integers from 2 to about 100; or

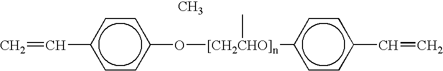

where: n is an integer from 4 to about 100; or

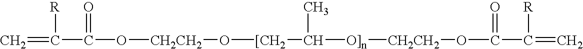

where: R is hydrogen or methyl; and

n is an integer from 4 to about 100.

According to one embodiment, preferred polypropylene glycol containing monomers include:

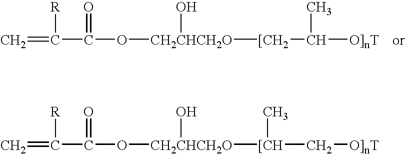

where: R is hydrogen or methyl;

T is a terminal group which is preferably hydrogen or an alkyl group;

n is an integer from 2 to about 100; or

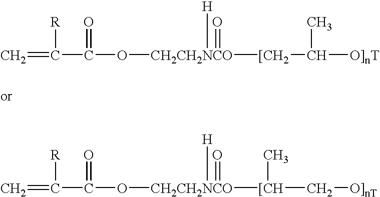

where: R is hydrogen or methyl;

T is a terminal group which is preferably hydrogen or an alkyl group;

n is an integer from 4 to about 100; or

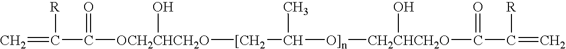

where: R is hydrogen or methyl;

n is an integer from 4 to about 100; or

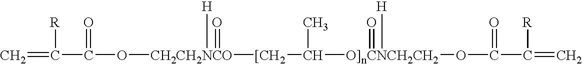

where: R is hydrogen or methyl;

n is an integer from 4 to about 100.

More preferred polypropylene glycol containing monomers include:

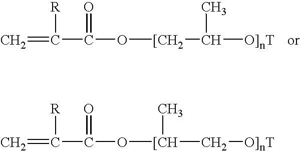

where: R is hydrogen or methyl;

T is a terminal group which is preferably hydrogen or an alkyl group;

n is an integer from 2 to about 100; or

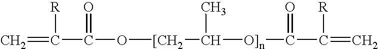

where: R is hydrogen or methyl; and

n is an integer from 2 to about 100.

In preparing the polymeric matrices, it is often preferable to form copolymers of the polypropylene glycol containing monomer with one or more comonomers. The drug release profile from these copolymer matrices can be altered considerably by the choice of comonomer(s). For example, use of a hydrophobic comonomer(s) with the polypropylene glycol containing monomer will form matrices that will be compatible with drugs that are hydrophobic. On the other hand, use of a hydrophilic comonomer(s) will produce matrices that are more compatible with hydrophilic drugs. The release profile of a drug from matrices described in this invention can also be altered by the degree of crosslinking. Matrices with higher degrees of crosslinking will retard the diffusion of the drug from the matrix, thus providing slower release rates.

The monomers which can be present in the polymers used to form the present devices can be any copolymerizable vinyl monomer. The following are representative groups of comonomers that can be employed and serve as examples only and are not intended to limit the scope of the invention.

Suitable comonomers include alkyl acrylates and methacrylates, especially C₁-C₂₀ alkyl acrylates and C₁-C₂₀ alkyl methacrylates, such as methyl methacrylate, ethyl methacrylate, methyl acrylate, butyl methacrylate, butyl acrylate, 2-ethylhexyl acrylate, and the like; alkonoic vinyl esters, especially C₁-C₆ alkanoic vinyl esters such as vinyl acetate, vinyl butyrate and the like; alkenes, especially C₁-C₈ alkenes, including ethylene, 1-butene, 1-hexene, and the like; styrenes, especially styrene and alpha-methyl styrene; vinyl ethers, especially C₁-C₆ alkyl vinyl ethers, including methyl vinyl ether, ethyl vinyl ether and butyl vinyl ether, and the like; dialkyl maleates, fumarates or itaconates, especially C₁-C₆ dialkyl maleates, fumarates or itaconates, including dimethyl maleate, dimethyl fumarate, diethyl maleate, dimethyl itaconate and the like; allyl ethers and esters, especially allyl C₁-C₆ alkyl ethers and allyl C₂-C₆ alkanoate esters, including allyl methyl ether, allyl ethyl ether, allyl acetate and the like; perfluoro C₃-C₆ alkyl acrylates or methacrylates; perfluoroalkoxylated bis-acrylates or -methacrylates; poly- or oligo-alkylsiloxane acrylates or methacrylates, and the like.

Also, minor amounts of a crosslinking agent, to alter drug release characteristics, stability and the mechanical properties of the polymer are generally employed. Suitable crosslinking agents include, for example, C₂-C₆ alkylene ether di-methacrylates and acrylates, e.g., ethylene glycol dimethacrylate, diethylene glycol dimethacrylate, triethylene glycol dimethacrylate, glycerine trimethacrylate; allyl acrylate or methacrylate, divinyl benzene, poly- or oligo-alkylsiloxane di-acrylate or -methacrylate, and the like.

Suitable hydrophilic comonomers are hydroxyl-substituted lower alkyl acrylates and methacrylates, acrylamide, methacrylamide, (lower alkyl)acrylamides and methacrylamides, N,N-dialkyl-acrylamides, ethoxylated acrylates and methacrylates, polyethyleneglycol-mono(meth)acrylates and polyethyleneglycolmonomethylether-(meth)acrylates, hydroxyl-substituted (lower alkyl)acrylamides and -methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine,vinylically unsaturated carboxylic acids having a total of 3to 5 carbon atoms, amino(lower alkyl)- (where the term “amino” also includes quaternary ammonium), mono(lower alkylamino)(lower alkyl) and di(lower alkylamino)(lower alkyl)acrylates and methacrylates, allyl alcohol and the like. Preference is given for example, to N-vinyl-2-pyrrolidone, acrylamide, dimethyl acrylamide, methacrylamide, hydroxyl-substituted lower alkyl acrylates and methacrylates, hydroxy-substituted (lower alkyl)acrylamides and -methacrylamides and vinylically unsaturated carboxylic acids having a total of 3 to 5 carbon atoms.

Suitable fluorinated monomers include 1,1,2,2-tetrahydroperfluorodecyl acrylates and methacrylates, 1,1,2,2-tetrahydroperfluorooctyl acrylate and methacrylate and 1,1,2,2-tetrahydroperfluorooctyl methacrylamide or acrylamide, hexafluoroisopropyl acrylate, hexafluoroisopropyl methacrylate, perfluorocylcohexyl methacrylate, and 2,3,4,5,6-pentafluoro-styrene; the acrylates and methacrylates of fluoroalkyl substituted amido-alcohols, such as of C₇F₁₅CON(C₂H₅)C₂HOH; of sulfonamido-alcohols, such as of C₈F₁₇C₈H₄SO₂N(CH₃)—C₄H₈OH and C₈C₁₇SO₂N(C₂H₅)—C₂H₄OH; of perfluoroether alcohols, such as of C₃F₇—O(C₃F₆O)₂CF(CF₃)CH₂OH or (CF₃)₂CFO(CF₂CF₂)₂—CH₂CH₂OH; an and methacrylate of fluorinated thioether alcohols of structure CF₃(CF₂)ⱼCH₂CH₂SCH₂CH₂CH₂OH; acrylates and methacrylates of sulfonamido-amines, such as of RⱼSO₂NH(CH₃)CH₂CH₂N(CH₃)—(CH₂)₃NH and RⱼCH₃SO₂NH(CH₂)₂; of amido-amines, such as of RⱼCONH(CH₂)₂NH₂; as well as the vinyl monomers obtained by reaction of these aforementioned fluorinated alcohols and amines with 2-isocyanatoethyl acrylate or methacrylate or m-isopropenyl-1,1-dimethylbenzyl isocyanate.

Suitable silicone containing vinyl monomers are oligosiloxanyl-silylalkyl acrylates and methacrylates containing from 2-10 Si-atoms. Typical representatives include: tris(trimethylsiloxy-silyl)propyl(meth)acrylate, triphenyldimethyl-disiloxanylmethyl(meth)acrylate, pentamethyl-disiloxanylmethyl(meth)acrylate, tertbutyl-tetramethyl-disiloxanylethyl (meth)acrylate, methyl-di(trimethylsiloxy)silylpropyl-glyceryl(meth)acrylate; pentamethyldi-siloxanyl-methyl methacrylate; heptamethyl-cyclotetrasiloxy methyl methacrylate; heptamethyl-cyclotetrasiloxy-propyl methacrylate; (trimethylsilyl)-decamethyl-pentasiloxy-propyl methacrylate; dodecamethyl pentasiloxypropyl methacrylate.

Polymerization of the polypropylene glycol containing monomers of this invention alone, or with comonomers, may be carried out by employing initiators which generate free-radicals on application of an activating energy as is conventionally used in the polymerization of ethylenically unsaturated monomers. Included among free-radical initiators are the conventional thermally activated initiators such as azo compounds, organic peroxides and organic hydroperoxides. Representative examples of such initiators include beiizoyl peroxide, tertiary-butyl perbenzoate, diisopropyl peroxydicarbonate, cumene hydroperoxide, azobis(isobutryonitrile), and the like. Generally, from about 0.01 to 5 percent by weight of thermal initiator is used.

UV-initiated polymerization is carried out using photoinitiators. Such initiators are well known and have been described, for example, in polymerization art, e.g., Chapter II of “Photochemistry” by Calvert and Pitts, John Wiley & Sons (1966). The preferred initiators are photoinitiators which facilitate polymerization when the composition is irradiated. Representative examples of such initiators include acyloin and derivatives thereof, such as benzoin, benzoin methyl ether, benzoin ethyl ether, benzoin isopropyl ether, benzoin isobutyl ether and α-methylbenzoin; diketones such as benzil and diacetyl, etc.; ketones such as acetophenone, α,α,α-tribromoacetophenone, α,α-diethoxyacetophenone (DEAP), 2-hydroxy-2-methyl-1-phenyl-1-propanone, o-nitro-α,α,α-tnibromoacetophenone, benzophenone and p,p′-tetramethyldiaminobenzophenone; α-acyloxime esters such as benzil-(O-ethoxycarbonyl)-α-monoxime; ketone/amine combinations such as benzophenone/N-methyldiethanolamine, benzophenone/tributylamine and benzophenone/Michler's ketone; and benzil ketals such as benzil dimethyl ketal, benzil diethyl ketal and 2,5-dichlorobenzil dimethyl ketal. Normally, the photoinitiator is used in amounts ranging from about 0.01 to 5% by weight of the total composition.

Visible light polymerization is carried out using initiators that are activated by visible light, especially blue light. Representative examples include ferrocenium salts, aryldiazonium salts, diaryliodonium salts and triarylsulfonium salts, camphorquinone systems and dye/co-initiator systems.

Polymerization can be carried out in bulk in a conventional manner or in the presence of a solvent. Solvents are usually required to compatibilize components, including the drug when present. The amount of solvent depends on the nature and relative amounts of comonomers and drug, if present. Useful solvents to carry out the polymerization includes ketones, like acetone, methyl ethyl ketone, methyl propyl ketone, methyl isobutyl ketone and cyclohexane; alcohols like methanol, ethanol, isopropanol or ethyl-cellosolve; ethers like ethylene glycol or diethylene glycol dimethyl ether; esters like ethyl acetate or isopropyl acetate; dimethyl sulfoxide; N-methylpyrrolidone; N,N-dimethylformamide; N,N-dimethylacetamide and the like.

The polymerization can be carried out in molds which can be formed of plastics, glass or metal or any other suitable material and can be any shape, for example, film, sheet or rod.

The monomer mixture can be polymerized as is, or it can be polymerized with the drug included. After the polymerization, the casting is removed from the mold and any solvent present is removed by conventional means.

In the case where the drug is not included in the polymerizaiton mixture, a drug loading step needs to be performed. This is generally accomplished by dissolving the drug in an appropriate solvent (e.g., one that swells the matrix polymer) and placing the matrix polymer in that solution to allow drug uptake. Once equilibrium is reached the matrix, loaded with drug, is then removed from the solvent and dried.

Suitable drugs or active agents that can be utilized with the present delivery devices include, by way of example only, but are not limited to:

Anti-infectives: such as antibiotics, including tetracycline, chlortetracycline, bacitracin, neomycin, polymyxin B, gramicidin, oxytetracycline, chloramphenicol, and erythromycin; sulfonamides, including sulfacetamide, sulfamethizole, sulfisoxazole; quinolones, including ofloxacin, norfloxacin, ciprofloxacin, sporfloxacin; aminoglycosides, including amikacin, tobramycin, gentamicin; cephalosporins; combinations of antibiotics; antivirals, including idoxuridine, trnfluridine, vidarabine cidofovir, foscarnet sodium, ganciclovir sodium and acyclovir; antifingals such as amphotericin B, nystatin, flucytosine, fluconazole, natamycin, miconazole and ketoconazole; and other anti-infectives including nitrofurazone and sodium propionate.

Antiallergeiics: such as antzoline, methapyriline, clilorpheniramine, pyrilamine and prophenpyridamine, emedastine, ketorolac, levocabastin, lodoxamide, loteprednol, naphazoline/antazoline, naphazoline/pheniramine, olopatadine and cromolyn sodium.

Anti-inflammatories: such as hydrocortisone, hydrocortisone acetate, dexamethasone, dexamethasone 21-phosphate, fluocinolone, medrysone, prednisolone, prednisolone 21-phosphate, prednisolone acetate, fluorometholone, fluorometholone acetate, meddrysone, loteprednol etabonate, rimexolone.

Nonsteroidal anti-inflammatories: such as flurbiprofen, suprofen, diclofenac, indomethacin, ketoprofen, and ketorolac.

Decongestants: such as phenylephrine, naphazoline, oxymetazoline, and tetrahydrazoline.

Miotics and anticholinesterases: such as pilocarpine, eserine talicylate, carbachol, diisopropyl fluorophosphate, phospholine iodide, and demecarium bromide.

Mydriatics: such as atropine sulfate, cyclopentolate; homatropine, scopolamine, tropicamide, eucatropine, and hydroxyamphetamine.

Furthennore, the following active agents are also useful in the present devices:

Antiglaucoma agents: such as adrenergics, including epinephrine and dipivefrin, epinephryl borate; β-adrenergic blocking agents, including levobunolol, betaxolol, metipranolol, timolol, carteolol; α-adrenergic agonists, including apraclonidine, clonidine, brimonidine; parasympathomimetics, including pilocarpine, carbachol; cholinesterase inhibitors, including isoflurophate, deniecarium bromide, echothiephate iodide; carbonic anhydrase inhibitors, including dichlorophenamide acetazolamide, methazolamide, dorzolalide, brinzolamide, dichlorphenamide; prostaglandins, including latanoprost, travatan, bimatoprost; diconosoids and combinations of the above, such as a p-adrenergic blocking agent with a carbonic anhydrase inhibitor.

Anticataract drugs: such as aldose reductase inhibitors including tolerestat, statol, sorbinil; antioxidants, including ascorbic acid, vitamin E; nutritional supplements, including glutathione and zinc.

Lubricants: such as glycerin, propylene glycol, polyethylene glycol and polyglycerins.

The following examples are merely illustrative of the present carriers for controlled delivery of an active agent and the examples should not be considered as limiting its scope in any way.

A key to the ingredients used in Examples 1 through 16 is given in Table 1.

TABLE 1

| CODE | DESCRIPTION | SOURCE | CAT. NO. |
|---|---|---|---|
| PPGM | Poly(propylene glycol) | Aldrich | 46,979-3 |
| | methacrylate | | |
| HEMA | Hydroxyethylmethacrylate | Aldrich | 47,702-9 |
| TRIS | Methylacyloxypropyltris | Gelest | SIM6487.6 |
| | (trimethylsiloxy)silane | | |
| AZ0 | 2,2′-azobisisobutyronitrile | Aldrich | 44,109-0 |
| DEXA | Dexamethasone | Sigma | D-1756 |
| DEXA-A | Dexamethasone 21-acetate | Sigma | D-1881 |
| DEXA-P | Dexamethasone 21-phosphate | Sigma | D-1159 |
| BME | Benzoin methyl ether | Aldrich | B870-3 |
| TIM | Timolol maleate | Sigma | T6394 |

### EXAMPLE 1

The following example details the purification of the monomers utilized in exemplary formulations for the present devices (e.g., carriers). Impurities and inhibitors are removed from the as-received monomers through adsorption onto alumina oxide. The procedure is as follows: Approximately 2.0 gm of alumina oxide, activated and basic, is added to a 100 ml wide mouth jar followed by addition of approximately 20.0 gm of monomer. A magnetic stir bar is added to the jar, the jar is capped, and the contents gently stirred for about two days. The purified monomer is recovered by filtration through a 0.45 micron syringe filter. The purified monomer is stored under refrigeration until use.

### EXAMPLE 2

The following procedure illustrates the formulation and polymerization of certain exemplary compositions. It should be understood that this is one of many processes that can be utilized in the practice of the present devices and should not be taken as limiting the invention.

Firstly, the initiator and drug are dissolved in an appropriate solvent. Secondly, the solution is then combined with the purified monomer(s) to form a clear solution. The formulation is then transferred to a small test tube, usually a lOmm x 75 mm test tube. The formulation is purged with nitrogen to remove oxygen. The tube is then stoppered and placed in a 50° C. water bath and the polymerization process is allowed about three days. At that time the polymer is removed from the tube and the solvent allowed to evaporate at room temperature for five to seven days. At that point the polymer/drug combination is ready for drug release studies.

### EXAMPLE 3

The following formulation represents a drug delivery polymer vehicle that is essentially “neutral” in its hydrophobic/hydrophilic character. The drug utilized in this example is dexamethasone, a relatively hydrophobic drug.

| Ingredient | Amount |
|---|---|
| PPGM | 2.5 ml |
| DEXA | 0.025 gm |
| AZ0 | 0.015 gm |
| Ethanol | 1.0 ml |

The PPGM was purified by the procedure detailed in Example 1 and polymerized by the method given in Example 2. The resulting polymer/drug composition was a clear, rubbery material.

### EXAMPLE 4

The following formulation represents a drug delivery polymer vehicle that is essentially “hydrophobic” in its character. The drug utilized in this example is dexamethasone, a relatively hydrophobic drug.

| Ingredient | Amount |
|---|---|
| PPGM | 1.75 ml |
| TRIS | 0.75 m. |
| DEXA | 0.025 gm |
| AZ0 | 0.015 gm |
| Ethanol | 1.0 ml |

The PPGM and TRIS were purified by the procedure detailed in Example 1 and polymerized by the method given in Example 2. The resulting 10 polymer/drug composition was a translucent, rubbery material.

### EXAMPLE 5

The following formulation represents a drug delivery polymer vehicle that is essentially “hydrophilic” in its character. The drug utilized in this example is dexametbasone, a relatively bydrophobic drug.

| Ingredient | Amount |
|---|---|
| PPGM | 2.25 ml |
| HEMA | 0.25 m. |
| DEXA | 0.025 gm |
| AZ0 | 0.015 gm |
| Methanol | 1.0 ml |

The PGMN and HEMA 1 and polymerized by the method given in Example 2. The resulting polymer/drug composition was a clear, rubbery material.

### EXAMPLE 6

The following formulation represents a drug delivery polymer vehicle that is essentially “neutral” in its character. The drug utilized in this example is dexamethasone phosphate, a very hydrophilic, water soluble drug.

| Ingredient | Amount |
|---|---|
| PPGM | 2.5 ml |
| DEXA-P | 0.025 gm |
| AZ0 | 0.015 gm |
| Methanol | 1.0 ml |

The PPGM was purified by the procedure detailed in Example 1 and polymerized by the method given in Example 2. The resulting polymer/drug composition was a translucent, rubbery material.

### EXAMPLE 7

The following formulation represents a drug delivery polymer vehicle that is essentially “hydrophobic” in its character. The drug utilized in this example is dexamethasone phosphate, a very hydrophilic, water soluble drug.

| Ingredient | Amount |
|---|---|
| PPGM | 1.75 ml |
| TRIS | 0.75 m. |
| DEXA-P | 0.025 gm |
| AZ0 | 0.015 gm |
| Methanol | 1.50 ml |

The PPGM and TRIS were purified by the procedure detailed in Example 1 and polymerized by the method given in Example 2. The resulting polymer/drug composition was a translucent, rubbery material.

### EXAMPLE 8

The following fonnulation represents a drug delivery polymer vehicle that is essentially “hydrophilic” in its character. The drug utilized in this example is dexamethasone phosphate, a very hydrophilic, water soluble drug.

| Ingredient | Amount |
|---|---|
| PPGM | 1.75 ml |
| HEMA | 0.75 m. |
| DEXA-P | 0.025 gm |
| AZ0 | 0.015 gm |
| Methanol | 1.50 ml |

The PPGM and HEMA were purified by the procedure detailed in Example 1 and polymerized by the method given in Example 2. The resulting polymer/drug composition was a translucent, rubbery material.

### EXAMPLE 9

The following example details the preparation of a polymer vehicle containing a high loading of a dispersed drug. The drug utilized in this example was dexamethasone phosphate, a water soluble compound.

| Ingredient | Amount |
|---|---|
| PPGM | 1.75 ml |
| TRIS | 0.75 m. |
| DEXA-P | 0.333 gm |
| AZ0 | 0.015 gm |
| BME | 0.005 |
| Methanol | 0.50 ml |

The PPGM and TRIS were purified by the procedure detailed in Example 1. The AZO and BME were dissolved in the methanol and then the PPGM was added, followed by the TRIS. The DEXA-P powder was then dispersed in the formulation with rapid agitation. The formulation was then placed in a 10 mm×75 mm test tube, quickly purged with nitrogen, stoppered and placed in a Rayonet photochemical (UV) reactor. After five minutes exposure to the UV source the sample was removed from the reactor. The formulation had polymerized to a rubbery gel with the drug uniformly dispersed within. The test tube was then placed in a 50° C. water bath for three days to complete the polymerization process. At that time the polymer was removed from the tube and the solvent allowed to evaporate at room temperature for two to seven days. The resulting polymer/drug composition was a white, rubbery material.

### EXAMPLE 10

The following example details the method utilized to monitor drug release form the polymer/drug compositions of this invention, more specifically those disclosed in Examples 3 through 9.

Solutions of dexamethasone, in a concentration range of 5 ppm to 1,000 ppm, were prepared in Unisol® 4 buffer (Unisol® 4 is a preservative-free pH-balanced saline solution manufactured by Alcon Laboratories). A UV scanning spectrometer was utilized to generate a calibration curve of concentration, in gm/ml, of dexamethasone (λₘₐₓ=242) versus absorbance.

A similar calibration curve was also generated for dexamethasone phosphate (λₘₐₓ=242). A sample of drug loaded polymer weighing between 100 and 150 mg and of similar shape was placed in a 4 ml vial. To the vial was added 1.0 ml of Unisol® 4 buffer. After 24 hours at room temperature, the sample was removed and placed in another 4 ml vial and covered with 1.0 ml of fresh Unisol® 4 buffer. The 24-hour release vial was capped, labeled and held for analysis. This procedure was repeated four more times to obtain 1-, 2-, 3-, 4- and 5-day release data. The sampling interval was then expanded to every 3 to 5 days. The release study was carried out for a total of about 60 days.

The drug release samples were analyzed by UV spectroscopy and absorbance readings converted to weight of drug via the calibration curve. A plot of cumulative weight of drug released versus time was generated.

### EXAMPLE 11

The following example illustrates the ability of the polymeric material compositions of this invention to deliver drug in a controlled manner. The drug release characteristics of the polymeric matrix produced in Example 3 were determined by the methodology detailed in Example 10. The cumulative release, in micrograms, was plotted against elapsed time in days. The results were normalized to 0.100 gm of sample weight for comparative purposes. It can be seen from the plot that drug is released at a rapid rate over the first 15 days, followed by a slower, more stable rate, up to 60 days.

Neutral Gel Matrix/Hydrophobic Dexamethasone

[image]

Gel containing 1% Dexamethasone with release rate normalized to 0.100 gm sample weight.

### EXAMPLE 12

The following example illustrates the ability of the polymeric material compositions of this invention to deliver drug in a controlled manner. The drug release characteristics of the polymeric matrix produced in Example 4 were determined by the methodology detailed in Example 10. The cumulative release, in micrograms, was plotted against elapsed time in days. The results were normalized to 0.100 gm of sample weight for comparative purposes. It can be seen from the plot that drug is released at a rapid rate over the first 15 days, followed by a slower, more stable rate, up to 60 days.

Hydrophilic Gel Matrix/Hydrophobic Dexamethasone

[image]

Gel containing 1% Dexamethasone with release rate normalized to 0.100 gm sample weight.

### EXAMPLE 13

The following example illustrates the ability of the polymeric material compositions of this invention to deliver drug in a controlled manner. The drug release characteristics of the polymeric matrix produced in Example 5 were determined by the methodology detailed in Example 10. The cumulative release, in micrograms, was plotted against elapsed time in days. The results were normalized to 0.100 gm of sample weight for comparative purposes. It can be seen from the plot that drug is released at a rapid rate over the first 10 days, followed by a slower, more stable rate, up to 30 days.

Hydrophilic Gel Matrix/Hydrophobic Dexamethasone

[image]

Gel containing 1% Dexamethasone with release rate normalized to 0.100 gm sample weight.

### EXAMPLE 14

The following example illustrates the ability of the polymeric material compositions of this invention to deliver drug in a controlled manner. The drug release characteristics of the polymeric matrix produced in Example 6 were determined by the methodology detailed in Example 10. The cumulative release, in micrograms, was plotted against elapsed time in days. The results were normalized to 0.100 gm of 10 sample weight for comparative purposes. It can be seen from the plot that drug is released at a rapid rate over the first 10 days, followed by a slower, more stable rate, up to 60 days.

Hydrophilic Gel Matrix/Hydrophobic Dexamethasone

[image]

Gel containing 1% Dexamethasone Phosphate with release rate normalized to 0.100 gm sample weight.

### EXAMPLE 15

The following example illustrates the ability of the polymeric material compositions of this invention to deliver drug in a controlled manner. The drug release characteristics of the polymeric matrix produced in Example 7 were determined by the methodology detailed in Example 10. The cumulative release, in rnicrograms, was plotted against elapsed time in days. The results were normalized to 0.100 gm of sample weight for comparative purposes. It can be seen from the plot that drug is released at a rapid rate over the first 10 days, followed by a slower, more stable rate, up to 40 days.

Hydrophilic Gel Matrix/Hydrophobic Dexamethasone

[image]

Phosphate

Gel containing 1% Dexamethasone Phosphate with release rate normalized to 0.100 gm sample weight.

### EXAMPLE 16

The following example illustrates the ability of the polymeric material compositions of this invention to deliver drug in a controlled manner. The drug release characteristics of the polymeric matrix produced in Example 8 were determined by the methodology detailed in Example 10. The cumulative release, in micrograms, was plotted against elapsed time in days. The results were normalized to 0.100 gm of sample weight for comparative purposes. It can be seen from the plot that after the first day, drug is released at a nearly constant rate over the 30 day test period.

Hydrophilic Gel Matrix/Hydrophobic Dexamethasone Phosphate

[image]

Gel containing 1% Dexamethasone Phosphate with release rate normalized to 0.100 gm sample weight.

### EXAMPLE 17

The following example illustrates the ability of the polymeric material compositions of this invention to deliver drug in a controlled manner. The drug release characteristics of the polymeric matrix produced in Example 9 were determined by the methodology detailed in Example 10. The cumulative release, in micrograms, was plotted against elapsed time in days. Because of the large amount of drug in the sample the results were normalized to 1.0 mg of sample weight for comparative purposes. It can be seen from the plot that most of the drug is released rapidly over the first 5 days, and the sample appears depleted of drug after about 10 days.

[image]

Gel containing 11.76% Dexamethasone Phosphate with release rate normalized to 0.001 gm sample weight.

### EXAMPLE 18

The following formulafion was prepared and polymerized. These compositions are representative of polymeric matrices useful for controlled drug delivery.

Samples

| | A | B | C | D |
|---|---|---|---|---|
| PPGM (ml) | 100 | 90 | 80 | 70 |
| TRIS (ml) | 0 | 10 | 20 | 30 |
| AZO (gm) | 0.6 | 0.6 | 0.6 | 0.6 |
| Methanol (ml) | 13.3 | 13.3 | 13.3 | 13.3 |

The PPGM and TRIS were purified by the procedure detailed in Example 1 and polymerized by the method given in Example 2. The resulting polymer compositions were transparent, rubbery materials.

### EXAMPLE 19

The following example illustrates the hydrophilic/hydrophobic balance of each of the formulations of Example 18. Equilibrium solvent content was determined by inumersing the dried polymeric matrix samples (from Example 18) in 20 ml of a selected solvent.

The samples were first weighed and then immersed in each of the solvents at room temperature. After 10 days the samples were at equilibrium and were weighed. The solvated weight and the dry weight were used to determine the bulk solvent content (solvent swell) in percent. Three samples were tested and the results averaged (Table 1).

TABLE 1
Bulk Solvent Content (Solvent Swell) in Percent

| | Weight Percent Swell | | | |
|---|---|---|---|---|
| Solvent | Sample A | Sample B | Sample C | Sample D |
| Water | 3.9 | 2.1 | 0.9 | 0.5 |
| Xylene | 129.4 | 146.9 | 169.6 | 190.9 |
| Acetonitrile | 132.5 | 122.2 | 117.5 | 87.7 |
| Isopropyl alcohol | 182.3 | 187.8 | 193.2 | 197.3 |
| Dichloromethane | 451.6 | 451.4 | 450.7 | 453.6 |

The varying hydrophilic/hydrophobic balance of above samples is quite evident when comparing water, xylene and acetonitrile. As the content of “TRIS” in the copolymer increases the water content dramatically decreases. Swell in acetonitrile, a very polar molecule, decreases as the TRIS content increases. On the other hand, xylene swell increases as the content of “TRIS” in the copolymer increases. There is a slight trend in swell with isopropanol and no trend with dichloromethane. The two polar solvents, water and acetonitrile, and the non-polar xylene provide solvent swells that are consistent with the hydrophilic/hydrophobic balance of each of the four samples.

### EXAMLE 20

This example illustrates one method for incorporating drugs into a polymeric matrix of Example 18.

The four polymeric matrices were loaded with timolol maleate by solvent swell and partitioning of the drug in the polymer. Samples of dried polymer matrix material weighing between 100 and 200 mg and of similar shape were placed in 20 ml glass vials with 10 ml of a 2.0 weight percent timolol maleate solution in isopropanol (IPA). The polymer matrix samples were then allowed to swell in the timolol maleate/IPA solutions for 15 days at room temperature to achieve equilibrium loading of the drug. The drug-loaded samples were then allowed to dry at room temperature for one week to remove all of the IPA. This was verified by drying to constant weight.

The amount of drug uptake was estimated by placing a sample of each drug-loaded polymer, weighing apporximately 80 mg, in 30 ml of IPA to extract the timolol. The samples were extracted at room temperature for 21 days. The amount of timolol extracted was estimated from UV absorbance values converted to micrograms via the calibration curve. It was determined that the absorbance values of timolol maleate in isopropanol closely approximates those of timolol maleate in buffer.

The total amount of timolol maleate contained in each polymeric matrix is presented below.

[image]

The amount of timolol maleate in each of the polymeric matrices decreases proportionally as the amount of TRIS is increased. This is expected because the timolo maleate is a salt (polar) and is more soluble in the more polar polymeric matrices. Increasing the TRIS content renders the polymeric matrix more hydrophobic.

### EXAMPLE 21

The following example details the method utilized to monitor drug release form the polymer/drug compositions of this invention, more specifically those disclosed in Example 20.

Solutions of timolol maleate, in a concentration range of 5 ppm to 1,000 ppm, were prepared in Unisol® 4 buffer (Unisol® 4 is a preservative-free pH-balanced saline solution manufactured by Alcon Laboratories). A UV scanning spectrometer was utilized to generate a calibration curve of concentration, in gm/ml, of timolol maleate (λₘₐₓ=290) verse absorbance.

A Sample of drug loaded polymer weighing between 100 and 150 mg and of similar shape was placed in a 4 ml vial. To the vial was added 1.0 ml of Unisol® 4 buffer. After 24 hours at room temperature, the sample was removed and placed in another 4 ml vial and covered with 1.0 ml of fresh Unisol® 4 buffer. The 24-hour released vial was capped, labeled and held for analysis. This procedure was repeated four more times to obtain 1-, 2-, 3-, 4- and 5-day release data. The sampling interval was then expanded to every 3 to 5 days. The release study was carried out for a total of about 60 days.

The drug release samples were analyzed by UV spectroscopy and absorbance readings converted to weight of drug via the calibration curve. A plot of cumulative weight of drug released versus time was generated.

### EXAMPLE 22

The following example illustrates the controlled release of timolol from the polymeric matrices described in Example 20. The timolol release characteristics of the polymeric matrices described in Example 20 were determined by the methodology established in Example 21. The cumulative release, in nucrograms, was plotted against elapsed time in days. The results were normalized to 0.1 00grn of sample weight for comparision purpose.

[image]

The most hydropilic polymer matrix, Sample A, contained the highest level of timolol and displayed fairly rapid release of the timolol over about 20 days. All of the timolol had been released after about 40 days. Sample B, the 90/10 copolymer, released timolol more slowly than Sample A, and after 100 days had released about 86% of its timolol content. Samples C and D presented the most interesting results. After an initial pulse in the first few days of release, the release rate progressively slows over the next 30 days and then displayed a rather constant release of timolol. In fact, from 40 to 100 days the release rate of Sample C was constant at 11.7 μg/day. Sample D had a constant rate of release of 8.3 μg/day. After 100 days of release Sample C had depleted 79% of its timolol loading while Sample D had depleted 66% of its timolol loading. These results are remarkable in that the rate of release becomes nearly constant and that this release occurs for 100 days and potentially longer.

What is claimed is:

1. A carrier for controlled delivery of an active agent, the carrier comprising:
a polymeric matrix formed of polymerized ethylenically unsaturated polypropylene glycol containing monomer which comprises 50% by weight of the polymeric matrix.

2. The carrier of claim 1, wherein the polypropylene glycol containing monomer has the formula: 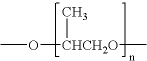
wherein n=2 to 100 and is derived from one or more monomers.

3. The carrier of claim 1, wherein the polypropylene glycol is derived from at least one of a monomer having a formula: 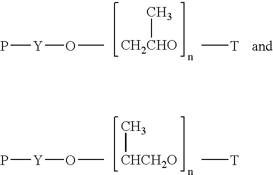
wherein:
P is an ethylenically unsaturated polymerizable group selected from the group consisting of: 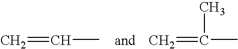
and Y is a spacer group selected from the group consisting of:
—CO—
—CONH—
—NHCO
—OCONH—
—CONHCO—
—CONHCONH—
—OCONHCO—
—NHCONHCO—
—NHCONH—
—CH₂—
—CH₂CH₂—
—CH₂CH₂CH₂—
—CH₂CH₂CH₂CH₂—
—C₆H₄—
—C₆H₄CH₂—
—COOCH₂CH(OH)CH₂—
—COOCH₂CH₂—
—COOCH₂CH₂OCH₂CH₂— and
—COOCH₂CH₂NHCO—
and T is a terminal group selected from the group consisting of hydrogen and an alkyl group; and
n is an integer from 2 to about 100.

4. The carrier of claim 1, wherein the polypropylene glycol is derived from at least one of a monomer having a formula:
and 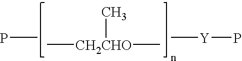
wherein
P is an ethylenically unsaturated polymerizable group selected from the group consisting of: 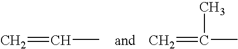
Wherein Y is a spacer group selected from the group consisting of:
—CO—
—CONH—
—NHCO—
—OCONH—
—CONHCO—
—CONHCONH—
—OCONHCO—
—NHCONHCO—
—NHCONH—
—CH₂—
—CH₂CH₂—
—CH₂CH₂CH₂—
—CH₂CH₂CH₂CH₂—
—C₆H₄—
—C₆H₄CH₂—
—COOCH₂CH(OH)CH₂—
—COOCH₂CH₂—
—COOCH₂CH₂OCH₂CH₂— and
—COOCH₂CH₂NHCO
wherein n is an integer from 4 to about 100.

5. The carrier of claim 1, wherein the polypropylene glycol is derived from at least one of a monomer having a formula: 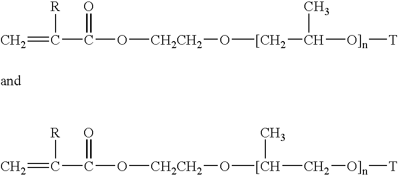
wherein: R is hydrogen or methyl;
T is a terminal group; and
n is an integer from 2 to about 100.

6. The carrier of claim 1, wherein the polypropylene glycol is derived from at least one of a monomer having a formula: 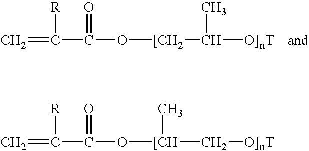
wherein: R is hydrogen or methyl;
T is a terminal group; and
n is an integer from 2 to about 100.

7. The carrier of claim 1, wherein the polypropylene glycol is derived from at least one of a monomer having a formula: 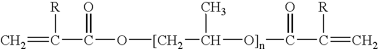
wherein: R is hydrogen or methyl; and
n is an integer from 2 to about 100.

8. The carrier of claim 1, wherein the ethylenically unsaturated polypropylene glycol containing monomers are copolymerized with other monomers to form a copolymer.

9. The carrier of claim 8, wherein the copolymer comprises 70% to 95% by weight of a monomer selected from the group consisting of: 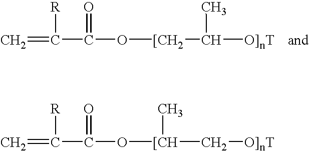
wherein: R is hydrogen or methyl;
T is a terminal group; and
n is an integer from 2 to about 100; and
5% to 30% by weight of the monomer having the structural formula: 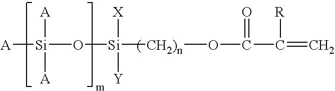
wherein X and Y are selected from the group consisting of C₁-C₅ alkyl groups, phenyl groups and Z groups, wherein Z is a group of the structure 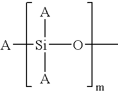
wherein A is selected from the group consisting of C₁-C₅ alkyl groups and phenyl groups; R is selected from the group consisting of methyl groups and hydrogen; m is an integer from one to five; and n is an integer from one to three.

10. The carrier of claim 8, wherein the copolymer comprises 70% to 95% by weight of the monomer polypropylene glycol monomethacrylate which is selected from one of: 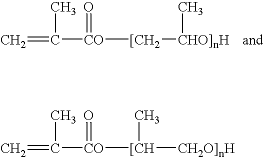
wherein: n is an integer from 2 to about 10; and the copolymer comprises 5% to 30% by weight of a monomer having the formula:

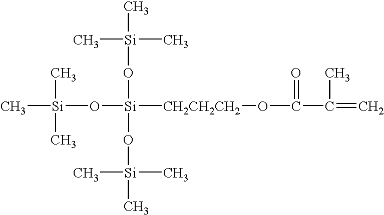

11. The carrier of claim 8, wherein the copolymer comprises 70% to 95% by weight of a monomer selected from the group consisting of 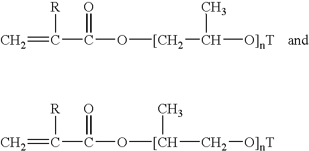
wherein: R is hydrogen or methyl;
T is a terminal group which is preferably hydrogen or an alkyl group; and
n is an integer from 2 to about 100; and the copolymer comprises 5% to 30% by weight of a monomer selected from the group consisting of

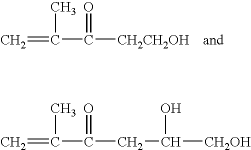

12. The carrier of claim 1, wherein the active agent is dissolved in the polymeric matrix.

13. The carrier of claim 1, wherein the active agent is dispersed throughout the polymeric matrix.

14. The carrier of claim 1, wherein the carrier is configured as an ocular insert.

15. The carrier of claim 1, wherein the active agent is selected from the group consisting essentially of anti-infectives, anti-allergenics, anti-inflammatories, decongestants, miotics, anti-cholinesterases, mydriatics, anti-glaucoma agents, and anti-cataract agents.

16. The carrier of claim 1, wherein the polypropylene glycol is derived completely from the monomers that contain ethylenically unsaturated polypropylene glycol.

17. The carrier of claim 1, wherein said carrier contains greater than 75% by weight of polypropylene glycol segments.
